(19) United States
(12) Patent Application Publication
(10) Pub. No.: US 2024/0172759 A1
(43) Pub. Date: May 30, 2024

(54) FORMULATION COMPRISING CHLORITE AND PERCARBONATE SALTS

(71) Applicant: ASEPTIC HEALTH, LLC, Nashville, TN (US)

(72) Inventors: Autumn RYAN, Nashville, TN (US); Lawrence MUNIE, Grayslake, IL (US)

(21) Appl. No.: 18/364,116

(22) PCT Filed: Feb. 2, 2022
(86) PCT No.: PCT/US2022/014986

§ 371 Date: Aug. 2, 2023

Related U.S. Application Data
(60) Provisional application No. 63/144,540, filed on Feb. 2, 2021.
(60) Provisional application No. 63/285,736, filed on Dec. 3, 2021.

(51) Int. Cl. A01N 59/00; A01N 25/02; A01P 1/00

(52) U.S. Cl. CPC A01N 59/00; A01N 25/02; A01P 1/00

(57) ABSTRACT

The present disclosure provides aqueous antimicrobial formulations comprising an aqueous solution comprising a chlorite salt in a concentration ranging from about 0.25% to about 25% by weight of the aqueous solution, a percarbonate salt in a concentration ranging from about 0.25% to about 25% by weight of the aqueous solution, and a surfactant in a concentration ranging from about 0.05% to about 50% by weight of the aqueous solution. The present disclosure further provides methods for using the antimicrobials formulations in a variety of settings.

### CROSS-REFERENCES TO RELATED APPLICATIONS

This application claims benefit of U.S. Patent Application No. 63/144,540 filed Feb. 2, 2021 entitled FORMULATION COMPRISING CHLORITE AND PERCARBONATE SALTS, and claims benefit of U.S. Patent Application No. 63/285,736 filed Dec. 3, 2021 entitled FORMULATION COMPRISING CHLORITE AND PERCARBONATE SALTS, which are both incorporated by reference in their entireties.

### TECHNICAL FIELD

The present disclosure generally relates to formulations comprising chlorite and percarbonate salts and methods of using such formulations for killing harmful pathogens, including bacteria, viruses, and fungi.

### TECHNICAL BACKGROUND

Clostridium difficile (C. difficile), Staphylococcus aureus (S. aureus), Escherichia coli (E. coli), Pseudomonas aeruginosa (P. aeruginosa), Enterobacter aerogenes (E. aerogenes), and other harmful bacteria can be found in a variety of environments including, but not limited to, medical, industrial, residential, food preparation environments. Exposure to these bacteria can cause illness, disease, and/or infection, particularly in medical settings where patients may have open wounds or lowered immune systems. Additionally, harmful viruses can spread easily in these environments, as seen in the SARS-Cov-2 pandemic. Finally, harmful fungi can abound in such environments, also leading to illnesses. While there are many available products that can kill such organisms efficiently, many of these products contain harmful chemicals that can be toxic to humans if ingested and/or irritating and harmful to the touch.

### BACKGROUND ART

Additionally, many bacteria, fungi, and viruses can be found on agricultural products such as plants, herbs, vegetables, fruits, cannabis, hemp, etc., and in meat, dairy, and poultry facilities. For instance E. coli has been frequently discovered in harmful quantities on lettuce plants. Additionally, powdery mildew is a fungus which can negatively affect various agricultural products. Conventional antimicrobial products cannot be applied to these types of products because they can be harmful to the agricultural product itself. Thus while conventional cleaning formulations may be effective at killing the bacteria and fungi on the agricultural product, conventional formulations can also potentially kill the underlying agricultural product. Agricultural products are also meant for human consumption, so toxic chemicals cannot safely be used in these settings. Peracetic acid (PAA) is currently used for removing bacteria and fungi from food products. But one disadvantage to PAA is that it can cause the discoloration of fruit. Accordingly, there is a need for alternative disinfecting agents for the agricultural industry.

Accordingly, formulations that are effective against a variety of microbes are still needed. Such formulations should be useful and safe to use across a variety of settings.

### SUMMARY OF THE INVENTION

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure advantageously provides antimicrobial formulations and methods for using them. In one embodiment, an antimicrobial formulation comprises an aqueous solution comprising a chlorite salt in a concentration ranging from about 0.25% to about 25% by weight of the aqueous solution, a percarbonate salt in a concentration ranging from about 0.25% to about 25% by weight of the aqueous solution, and a surfactant in a concentration ranging from about 0.05% to about 25% by weight of the aqueous solution.

In some embodiments, the chlorite salt is chosen from ammonium chlorite, calcium chlorite, magnesium chlorite, potassium chlorite, sodium chlorite, and any combination thereof. In other embodiments, the chlorite salt comprises the sodium chlorite.

In some embodiments, the concentration of the chlorite ranges from about 0.05% to about 1%, 0.25% to about 0.9%, 0.25% to about 0.8%, 0.25% to about 0.7%, 0.25% to about 0.6%, 0.25% to about 0.5%, about 0.5% to about 0.9%, about 0.5% to about 0.8%, about 0.5% to about 0.7%, about 0.5% to about 0.6%, about 0.05% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20% about 10% to about 15%, or about 15% to about 20% by weight of the aqueous solution.

In some embodiments, the percarbonate salt is chosen from ammonium percarbonate, calcium percarbonate, magnesium percarbonate, potassium percarbonate, sodium percarbonate, and any combination thereof. In some embodiments, the percarbonate salt comprises the sodium percarbonate.

In some embodiments, the concentration of the percarbonate ranges from about 0.25% to about 1%, 0.25% to about 0.9%, 0.25% to about 0.8%, 0.25% to about 0.7%, 0.25% to about 0.6%, 0.25% to about 0.5%, about 0.5% to about 0.9%, about 0.5% to about 0.8%, about 0.5% to about 0.7%, about 0.5% to about 0.6%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20% about 10% to about 15%, or about 15% to about 20% by weight of the aqueous solution.

In some embodiments, the surfactant comprises lauramine oxide, sodium dodecylbenzene sulfonate, sodium decyl sulfate, sodium docecyl sulfate, sodium octyl sulfonate, sodium xylene sulfonate, magnesium lauryl sulfate, sodium lauryl sulfate, C₉-C₁₁ ethoxylated alcohols, or any combination thereof. In some embodiments, the surfactant comprises the sodium dodecylbenzene sulfonate, the C₉-C₁₁ ethoxylated alcohols, or a combination thereof.

In some embodiments, the concentration of the surfactant ranges from about 0.05% to about 50%, about 0.05% to about 1%, 0.05% to about 0.9%, 0.05% to about 0.8%, 0.05% to about 0.7%, 0.05% to about 0.6%, 0.05% to about 0.5%, about 0.05% to about 0.4%, about 0.05% to about 0.3%, about 0.05% to about 0.2%, about 0.05% to about 0.1, about 0.1% to about 1%, 0.1% to about 0.9%, 0.1% to about 0.8%, 0.1% to about 0.7%, 0.1% to about 0.6%, 0.1% to about 0.5%, about 0.1% to about 0.4%, about 0.1% to about 0.3%, about 0.1% to about 0.2%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, about 15% to about 20%, about 1% to about 50%, about 5% to about 50%, about 10% to about 50%, about 15% to about 50%, about 20% to about 50%, or about 25% to about 50% by weight of the aqueous solution.

In some embodiments, the antimicrobial formulation, further comprises a stabilizer in an amount ranging from about 10 mM to about 2500 mM. In some embodiments, the stabilizer comprises an acetate salt, a bicarbonate salt, a carbonate salt, a metasilicate salt, a citrate salt, or any combination thereof. In further embodiments, the stabilizer comprises ammonium acetate, calcium acetate, magnesium acetate, potassium acetate, sodium acetate, ammonium bicarbonate, calcium bicarbonate, magnesium bicarbonate, potassium bicarbonate, sodium bicarbonate, ammonium carbonate, calcium carbonate, magnesium carbonate, potassium carbonate, sodium carbonate, ammonium metasilicate, calcium metasilicate, magnesium metasilicate, potassium metasilicate, sodium metasilicate, sodium citrate, potassium citrate, or any combination thereof. In some embodiments, the stabilizer comprises potassium acetate, sodium acetate, potassium bicarbonate, sodium bicarbonate, potassium carbonate, sodium carbonate, potassium metasilicate, sodium metasilicate, or any combination thereof. In some embodiments, the stabilizer comprises sodium bicarbonate, sodium carbonate, or a combination thereof.

In some embodiments, the concentration of the stabilizer ranges from about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60, about 40 mM to about 50 mM, about 50 mM to about 100 mM, about 50 mM to about 90 mM, 50 mM to about 80 mM, 50 mM to about 70 mM, 50 mM to about 60 mM, 60 mM to about 100 mM, 60 mM to about 90 mM, 60 mM to about 80 mM, 60 mM to about 70 mM, 70 mM to about 100 mM, 70 mM to about 90 mM, 70 mM to about 80 mM, 80 mM to about 100 mM, 80 mM to about 90 mM, or 90 mM to about 100 mM, about 5 mM to about 2500 mM, about 10 mM to about 2500 mM, about 100 mM to about 2500 mM, about 500 mM to about 2500 mM, about 1000 mM to about 2500 mM, or about 2000 mM to about 2500 mM.

In certain embodiments, the antimicrobial formulation further comprises a buffer. In some embodiments, the buffer comprises acetic acid, citric acid, sodium acetate, potassium acetate, sodium carbonate, potassium carbonate, sodium bicarbonate, potassium bicarbonate, sodium citrate, potassium citrate, or any combination thereof.

In some embodiments, the pH of the antimicrobial formulation ranges from about 7 to about 10, about 7.5 to about 9.5, about 8 to about 9.5, about 8.5 to about 9.5, about 8 to about 9, or about 9 to about 9.5. In certain embodiments, the pH is about 9.

In some embodiments, the antimicrobial formulation further comprises at least one of a fragrance, a colorant, a sudsing agent, an aerosolizing agent, or a thickener.

In some embodiments, the antimicrobial formulation comprises about 0.25% to about 1% of the chlorite by weight of the formulation, about 0.25% to about 1% by weight of the percarbonate by weight of the formulation, and about 0.1% to about 0.5% of the surfactant by weight of the formulation.

In other embodiments, the antimicrobial formulation comprises about 10 to about 25% of the chlorite by weight of the formulation, about 10% to about 25% by weight of the percarbonate by weight of the formulation, and about 5% to about 25% of the surfactant by weight of the formulation.

In some embodiments the antimicrobial formulation is greater than 99.5%, greater than 99.9% effective, or greater than 99.99% effective against a virus, a bacterium, or a fungus.

In some embodiments, the antimicrobial formulation is effective against a virus chosen from Adenovirus Type 2, Avian Influenza A Virus, Bovine Viral Diarrhea Virus, Canine Coronavirus, Canine Distemper Virus, Canine Parvovirus, Coxsackie Virus, Cytomegalovirus, Duck Hepatitis B Virus, Feline Calicivirus, Feline Coronavirus, Feline Panleukopenia Virus, Hepatitis A Virus, Hepatitis B Virus, Hepatitis C Virus, Herpes Simplex Virus Type 1, Herpes Simplex Virus Type 2, Human Coronavirus, Human Immunodeficiency Virus Type 1, Influenza Virus Type A, Influenza Virus Type B, Norovirus, Parainfluenza Type 1, Parainfluenza Type 3, Poliovirus, Respiratory Syncytial Virus, Rhinovirus Type 17, Rhinovirus Type 37, Rotavirus, Rubella Virus, SARS-CoV-2, Varicella-Zoster Virus, and any combination thereof.

In some embodiments, the antimicrobial formulation is effective against a bacteria chosen Clostridium difficile, Staphylococcus aureus, Escherichia coli, Pseudomonas aeruginosa, Enterobacter sp. (e.g., E. aerogenes), Acinetobacter baumanni, Bordetella pertussis, E. coli, carbapenem-resistant E. coli, Klebsiella pneumonia, Legionella. pneumophila, Listeria monocytogenes, methicillin resistant Staphylococcus aureus, Pseudomonas aeruginosa, Salmonella enterica, Streptococcus sp, Trichophyton interdigitale, Enterococcus sp (e.g., vancomycin-resistant E. faecalis), Bordetella Bronchiseptica, Shigella dysenteriae, and any combination thereof.

In some embodiments, the antimicrobial formulation is effective against a fungus chosen from Alternaria alternata, Aspergillus fumigatus, Aspergillus niger, Candida albicans, Penicillium sp, Stachybotrys chararum, Trichophyton mentagrophytes, Trichophyton rubrum, Penicillium digitatum, Geotrichum citri-aurantii, Monilinia fructicola, Botrytis cinerea, and any combination thereof.

The present disclosure further provides methods for using the aforementioned antimicrobial formulations. In some embodiments, a method for disinfecting an object comprises applying the antimicrobial formulation to the object. In some embodiments, the object comprises a hard surface or a soft surface. In some embodiments, the surface comprises a food contact surface. In some embodiments, the surface is a textile.

In some embodiments, the surface is an agricultural product. In some embodiments, the agricultural product comprises a plant, such as cannabis. In some embodiments, the agricultural product comprises meat or poultry.

In some embodiments, the object is contaminated with a bacteria, a virus, a fungus, or any combination thereof, and the application of the antimicrobial formulation kills at least 99.5%, at least 99.9%, or at least 99.99% of the bacteria, virus, fungus, or any combination thereof.

Numerous other objects, advantages and features of the present disclosure will be readily apparent to those of skill in the art upon a review of the following detained description.

### BRIEF DESCRIPTION OF THE FIGURES

FIG. 1 is a table showing MIC values for different antimicrobial solutions, ther MIC value being the lowest concentration of an antimicrobial agent at which a test microorganism will not grow.

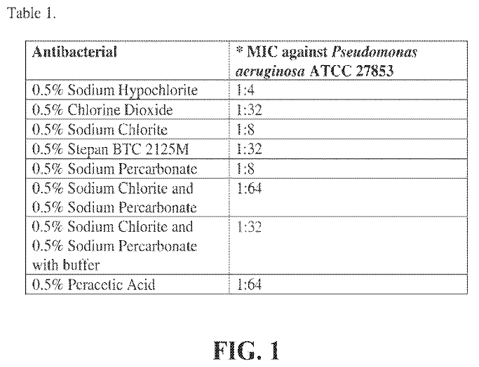

### MODES FOR CARRYING OUT THE INVENTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

The qualifier “about” as used herein regarding contact time can describe a tolerance of up to five seconds around the stated contact time. The qualifier “about,” when used regarding chlorine dioxide concentration describes a tolerance of up to five percent around the stated concentration.

### Antimicrobial Formulations

The present disclosure advantageously provides antimicrobial formulations that are remarkably effective at killing a variety of pathogens, including bacteria, viruses, and fungi, while also being non-corrosive and non-irritating to humans or animals. The present formulations include a chlorite salt and a percarbonate salt. While not being bound by theory, it is believed that the chlorite salt and percarbonate salt act synergistically to provide enhanced antimicrobial effects that would be unexpected if merely considering the additive effects of these agents.

In one embodiment, an antimicrobial formulation comprises an aqueous solution comprising a chlorite salt in a concentration ranging from about 0.25% to about 25% by weight of the aqueous solution, a percarbonate salt in a concentration ranging from about 0.25% to about 25% by weight of the aqueous solution, and a surfactant in a concentration ranging from about 0.05% to about 25% by weight of the aqueous solution.

In some embodiments, the chlorite salt is chosen from ammonium chlorite, calcium chlorite, magnesium chlorite, potassium chlorite, sodium chlorite, and any combination thereof. In other embodiments, the chlorite salt comprises the sodium chlorite.

In some embodiments, the concentration of the chlorite ranges from about 0.25% to about 1%, 0.25% to about 0.9%, 0.25% to about 0.8%, 0.25% to about 0.7%, 0.25% to about 0.6%, 0.25% to about 0.5%, about 0.5% to about 0.9%, about 0.5% to about 0.8%, about 0.5% to about 0.7%, about 0.5% to about 0.6%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20% about 10% to about 15%, or about 15% to about 20% by weight of the aqueous solution.

In some embodiments, the percarbonate salt is chosen from ammonium percarbonate, calcium percarbonate, magnesium percarbonate, potassium percarbonate, sodium percarbonate, and any combination thereof. In some embodiments, the percarbonate salt comprises the sodium percarbonate.

In some embodiments, the concentration of the percarbonate ranges from about 0.25% to about 1%, 0.25% to about 0.9%, 0.25% to about 0.8%, 0.25% to about 0.7%, 0.25% to about 0.6%, 0.25% to about 0.5%, about 0.5% to about 0.9%, about 0.5% to about 0.8%, about 0.5% to about 0.7%, about 0.5% to about 0.6%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20% about 10% to about 15%, or about 15% to about 20% by weight of the aqueous solution.

The present formulations also comprise a surfactant. The surfactant in some embodiments is non-reactive with the chlorite salt. In particular embodiments, the surfactant is non-amine surfactant. In still other embodiments the surfactant can be any suitable surfactant that helps decrease the surface tension of the antimicrobial formulation to allow for easier dispersion of the antimicrobial compound on a surface or product of interest. In some embodiments, the surfactant can be an alkoxylated non-ionic surfactant, such as ethoxylated alcohols. In some embodiments, the ethoxylated alcohols comprise C₆-C₂₀ ethoxylated alcohols, while in other embodiments, the ethoxylated alcohols comprise C₉-C₁₁ ethoxylated alcohols, including those surfactants sold under the brand name Tomadol®, such as Tomadol 900. In other embodiments, the surfactant can include nonyl phenol ethoxylates, nonyl phenol propoxylates, or linear alkoxylated C₆-C₂₀ alcohols (4 mol-15 mol EO or PO). In some embodiments, the surfactant comprises lauramine oxide (e.g., Ammonyx® LO, sodium dodecylbenzene sulfonate (e.g., Bio-Soft® D-40), sodium decyl sulfate (e.g., Polystep® B-25), sodium octyl sulfonate (e.g., Polystep® B-29), sodium xylene sulfonate (e.g., Stepanate® SXS), magnesium lauryl sulfate (e.g., Stepanol® Mg), or sodium lauryl sulfate (e.g., Stepanol® WA-Extra K).

In some embodiments, the surfactant comprises the sodium dodecylbenzene sulfonate (e.g. Bio-Soft® D-40), while in other embodiments, the surfactant comprises the C₉-C₁₁ ethoxylated alcohols (e.g., Tomadol® 900). In other embodiments, the surfactant comprises the sodium dodecylbenzene sulfonate and the C₉-C₁₁ ethoxylated alcohols.

In some embodiments, the concentration of the surfactant ranges from 0.05% to about 50%, about 0.05% to about 1%, 0.05% to about 0.9%, 0.05% to about 0.8%, 0.05% to about 0.7%, 0.05% to about 0.6%, 0.05% to about 0.5%, about 0.05% to about 0.4%, about 0.05% to about 0.3%, about 0.05% to about 0.2%, about 0.05% to about 0.1, about 0.1% to about 1%, 0.1% to about 0.9%, 0.1% to about 0.8%, 0.1% to about 0.7%, 0.1% to about 0.6%, 0.1% to about 0.5%, about 0.1% to about 0.4%, about 0.1% to about 0.3%, about 0.1% to about 0.2%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, about 15% to about 20%, about 1% to about 50%, about 5% to about 50%, about 10% to about 50%, about 15% to about 50%, about 20% to about 50%, or about 25% to about 50% by weight of the aqueous solution.

The antimicrobial formulations may, in some embodiments, further comprise a stabilizer in an amount ranging from about 10 mM to about 2500 mM. In some embodiments, the stabilizer comprises an acetate salt, a bicarbonate salt, a carbonate salt, a metasilicate salt, a citrate salt, or any combination thereof. In further embodiments, the stabilizer comprises ammonium acetate, calcium acetate, magnesium acetate, potassium acetate, sodium acetate, ammonium bicarbonate, calcium bicarbonate, magnesium bicarbonate, potassium bicarbonate, sodium bicarbonate, ammonium carbonate, calcium carbonate, magnesium carbonate, potassium carbonate, sodium carbonate, ammonium metasilicate, calcium metasilicate, magnesium metasilicate, potassium metasilicate, sodium metasilicate, sodium citrate, potassium citrate, or any combination thereof. In some embodiments, the stabilizer comprises potassium acetate, sodium acetate, potassium bicarbonate, sodium bicarbonate, potassium carbonate, sodium carbonate, potassium metasilicate, sodium metasilicate, or any combination thereof. In some embodiments, the stabilizer comprises sodium bicarbonate, sodium carbonate, or a combination thereof.

In some embodiments, the concentration of the stabilizer ranges from about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60, about 40 mM to about 50 mM, about 50 mM to about 100 mM, about 50 mM to about 90 mM, 50 mM to about 80 mM, 50 mM to about 70 mM, 50 mM to about 60 mM, 60 mM to about 100 mM, 60 mM to about 90 mM, 60 mM to about 80 mM, 60 mM to about 70 mM, 70 mM to about 100 mM, 70 mM to about 90 mM, 70 mM to about 80 mM, 80 mM to about 100 mM, 80 mM to about 90 mM, or 90 mM to about 100 mM, about 5 mM to about 2500 mM, about 10 mM to about 2500 mM, about 100 mM to about 2500 mM, about 500 mM to about 2500 mM, about 1000 mM to about 2500 mM, or about 2000 mM to about 2500 mM.

In certain embodiments, the antimicrobial formulation further comprises a buffer. In some embodiments, the buffer comprises acetic acid, citric acid, sodium acetate, potassium acetate, sodium carbonate, potassium carbonate, sodium bicarbonate, potassium bicarbonate, sodium citrate, potassium citrate, or any combination thereof.

In the composition disclosed herein, the additional ingredients in the compound other than the chlorite salt are such that the chlorite salt does not react with any additional ingredients to form any substantial amount of chlorine dioxide. As such, the concentration of chlorine dioxide is less than 0.01% by weight of the antimicrobial formulation. In other embodiments, the concentration of chlorine dioxide can be less than 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% by weight of the aqueous solution. Similarly, while certain weak acids such as acetic acid, citric acid, etc., can be utilized as a buffer for the solution, these acids can be present in concentrations that do not cause a substantial reaction of the chlorite salt to chlorine dioxide, and maintain the concentrations of chlorine dioxide in solution to values less than disclosed herein. In some embodiments, the concentration of acid in the antimicrobial formulation can be less than 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, or 0.001%. In other embodiments, no acid is present in solution and the concentration of chlorine dioxide in solution is substantially zero.

In some embodiments, the pH of the antimicrobial formulation ranges from about 7 to about 10, about 7.5 to about 9.5, about 8 to about 9.5, about 8.5 to about 9.5, about 8 to about 9, or about 9 to about 9.5. In certain embodiments, the pH is about 9.

The antimicrobial formulations may be provided in a ready-to-use form or as a concentrate. In some embodiments the antimicrobial formulation is ready-to-use and comprises about 0.25% to about 1% of the chlorite by weight of the formulation, about 0.25% to about 1% by weight of the percarbonate by weight of the formulation, and about 0.1% to about 0.5% of the surfactant by weight of the formulation. In some embodiments, a ready-to-use formulation further comprises a stabilizer in an amount ranging from about 10 mM to about 100 mM.

In other embodiments, the antimicrobial formulation may be provided as a concentrated formulation and comprises about 10 to about 25% of the chlorite by weight of the formulation, about 10% to about 25% by weight of the percarbonate by weight of the formulation, and about 5% to about 25% of the surfactant by weight of the formulation. In some embodiments, a concentrated formulation further comprises a stabilizer in an amount ranging from about 1000 mM to about 2500 mM. The concentrated formulation may be diluted by a user prior to application to a desired site.

The above-described formulations contain a combination of chlorite salt and percarbonate salt as a microbial agent. In certain embodiments, the chlorite and percarbonate salts are the only antimicrobial agents in the formulation. Thus, in some embodiments, the formulation comprises an antimicrobial agent, wherein the antimicrobial agent consists of the chlorite salt and the percarbonate salt. In some embodiments, the antimicrobial agent consists essentially of the chlorite salt and the percarbonate salt. As used herein, “consists essentially of the chlorite salt and the percarbonate” means that the chlorite and percarbonate salts provide all or substantially all of the antimicrobial activity in the formulation. In other embodiments, the formulations may contain additional antimicrobial agents.

The antimicrobial formulations described herein have significant antimicrobial activity against a variety of pathogens. In some embodiments the antimicrobial formulation is greater than 99.5%, greater than 99.9% effective, greater than 99.99%, greater than 99.999%, or greater than 99.9999% effective against a virus, a bacterium, or a fungus. Moreover, in some embodiments, the antimicrobial formulation is greater than 99.5%, greater than 99.9% effective, greater than 99.99%, greater than 99.999%, or greater than 99.9999% effective against a virus, a bacterium, or a fungus after a contact time of less than 10 minutes, less than five minutes, less than 120 seconds, less than 60 seconds, less than 45 seconds, or less than 30 seconds.

In some embodiments, the antimicrobial formulation is effective against a virus chosen from Adenovirus Type 2, Avian Influenza A Virus, Bovine Viral Diarrhea Virus, Canine Coronavirus, Canine Distemper Virus, Canine Parvovirus, Coxsackie Virus, Cytomegalovirus, Duck Hepatitis B Virus, Feline Calicivirus, Feline Coronavirus, Feline Panleukopenia Virus, Hepatitis A Virus, Hepatitis B Virus, Hepatitis C Virus, Herpes Simplex Virus Type 1, Herpes Simplex Virus Type 2, Human Coronavirus, Human Immunodeficiency Virus Type 1, Influenza Virus Type A, Influenza Virus Type B, Norovirus, Parainfluenza Type 1, Parainfluenza Type 3, Poliovirus, Respiratory Syncytial Virus, Rhinovirus Type 17, Rhinovirus Type 37, Rotavirus, Rubella Virus, SARS-CoV-2, Varicella-Zoster Virus, and any combination thereof.

In some embodiments, the antimicrobial formulation is effective against a bacteria chosen Clostridium difficile, Staphylococcus aureus, Escherichia coli, Pseudomonas aeruginosa, Enterobacter sp. (e.g., E. aerogenes), Acinetobacter baumanni, Bordetella pertussis, E. coli, carbapenem-resistant E. coli, Klebsiella pneumonia, Legionella. pneumophila, Listeria monocytogenes, methicillin resistant Staphylococcus aureus, Pseudomonas aeruginosa, Salmonella enterica, Streptococcus sp, Trichophyton interdigitale, Enterococcus sp (e.g., vancomycin-resistant E. faecalis), Bordetella Bronchiseptica, Shigella dysenteriae, and any combination thereof.

In some embodiments, the antimicrobial formulation is effective against a fungus chosen from Alternaria alternata, Aspergillus fumigatus, Aspergillus niger, Candida albicans, Penicillium sp, Stachybotrys chararum, Trichophyton mentagrophytes, Trichophyton rubrum, Penicillium digitatum, Geotrichum citri-aurantii, Monilinia fructicola, Botrytis cinerea, and any combination thereof.

The antimicrobial formulations described herein can be formulated for a variety of applications, including domestic cleaning, industrial cleaning, health care settings, personal care, food service settings, food contact settings, and agricultural settings. For example, the compositions can be formulated in ready to use or concentrated formulations for household cleaning, such as kitchen, bathroom, hard floors, carpeting, rug, laundry, and the like. The formulations may be combined with other commonly used household cleaners, soaps and detergents in a variety of packaging, such as pump bottles, spray bottles, aerosols, pourable bottles, buckets, etc.

Formulations for industrial uses, such as public restrooms, food service settings, hospitals, nursing care facilities, schools, day cares, dentist offices, veterinary settings, and agricultural settings also may be provided in ready to use or concentrated formulations in a variety of packing, including, pump bottles, spray bottles, aerosols, pourable bottles, buckets, etc. Such formulations also may contain additional ingredients used in the cleaning industry, such as detergents, soaps, and the like.

Formulations may also be prepared for medical use, such as in cleaning or sterilizing surgical units, tools, and medical devices.

In some embodiments, the antimicrobial composition may be formulated for personal care products, such as sanitizing gels, soaps, body washes, face washes, shampoos, lotions, wipes, etc. In one embodiment, the antimicrobial formulation can be formulated as a hand care product, such as a disinfecting gel, soap, spray, wipe, or lotion. The concentration of the active antimicrobial ingredients, the chlorite and percarbonate, can be in concentrations as little as 0.05%. Having a lower concentration of active ingredients can help reduce the occurrence of hand irritation or sensitivity while still providing adequate antimicrobial properties. In some embodiments, the personal care formulation can include an emollient. The emollient compound may be, in one embodiment, glycerin. In other embodiments, the emollient comprise glycerin shea butter, cocoa butter, lanolin, or any combination thereof. The hand care formulation may be advantageously packaged for various uses, such as in dispensers for health care setting, public restroom settings, personal dispensing bottles for travel, etc.

Depending on the particular intended use for the formulation, it may further comprise, in some embodiments, at least one of a fragrance, a colorant, a sudsing agent, an aerosolizing agent, detergent, and/or a thickener.

In some embodiments, the antimicrobial composition or formulation can be incorporated into an antimicrobial wipe. The wipe can include a solution of chlorite and percarbonate (in one embodiment sodium chlorite and sodium percarbonate) in any of the concentrations or combination of concentrations disclosed herein. The solution can be combine in a canister with a volume of substrates or wipes to form an antimicrobial wipe canister or dispenser for administering the antimicrobial composition. The antimicrobial formulation for use of the wipes can also include one or more of the various surfactants, stabilizers, buffers, etc. described herein, and in any of the concentrations disclosed herein. In one embodiment, the weight concentration of sodium chlorite is between about 0.05% and about 0.75% and the weight concentration of sodium percarbonate is between about 0.05% and about 0.7%, in water having a concentration of about 98.55%-99.9%. In some embodiments, the concentration of each of the sodium chlorite and sodium percarbonate can be between about 0.05% to about 0.6%, about 0.05% to about 0.5%, about 0.05% to about 0.4%, about 0.05% to about 0.2%, about 0.05% to about 0.15%, about 0.8% to about 0.2%, about 0.1% to about 0.15%, about 0.1% to about 0.75%, about 0.2% to about 0.75%, about 0.3% to about 0.75%, about 0.4% to about 0.75%, about 0.5% to about 0.75%, about 0.6% to about 0.75%, about 0.2% to about 0.7%, about 0.2% to about 0.6%, or about 0.3% to about 0.5% by weight.

In some embodiments, the antimicrobial composition or formulation can be in the form of a shampoo, body wash, face wash, or vanishing cream. In some embodiments, the concentration of each of the sodium chlorite and sodium percarbonate can be between about 0.05% to about 0.75%, about 0.05% to about 0.7%, about 0.05% to about 0.6%, about 0.05% to about 0.5%, about 0.05% to about 0.4%, about 0.05% to about 0.2%, about 0.05% to about 0.15%, about 0.8% to about 0.2%, about 0.1% to about 0.15%, about 0.1% to about 0.75%, about 0.2% to about 0.75%, about 0.3% to about 0.75%, about 0.4% to about 0.75%, about 0.5% to about 0.75%, about 0.6% to about 0.75%, about 0.2% to about 0.7%, about 0.2% to about 0.6%, or about 0.3% to about 0.5% by weight. The shampoo, body wash, and face wash applications can also include one or more surfactants ranging in total concentration from about 2% to about 25%, about 2% to about 25% by weight of the solution. In some embodiments, the concentration of the surfactant can range from about 0.05% to about 50%, about 0.05% to about 1%, 0.05% to about 0.9%, 0.05% to about 0.8%, 0.05% to about 0.7%, 0.05% to about 0.6%, 0.05% to about 0.5%, about 0.05% to about 0.4%, about 0.05% to about 0.3%, about 0.05% to about 0.2%, about 0.05% to about 0.1, about 0.1% to about 1%, 0.1% to about 0.9%, 0.1% to about 0.8%, 0.1% to about 0.7%, 0.1% to about 0.6%, 0.1% to about 0.5%, about 0.1% to about 0.4%, about 0.1% to about 0.3%, about 0.1% to about 0.2%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, about 15% to about 20%, about 1% to about 50%, about 5% to about 50%, about 10% to about 50%, about 15% to about 50%, about 20% to about 50%, or about 25% to about 50% by weight of the aqueous solution. In some embodiments, the one or more surfactants can include, but are not limited to, one or more of Sodium Laureth Sulfate, Cocamidopropyl betaine, Sodium Lauryl Sulfate, Cocamide MEA, Cocamide MIPA, and/or Cocamidopropyl betaine, The shampoo, body wash, and/or face wash applications can also include an emollient, humectant, or other moisturizing agent including but limited to glycerin, shea butter, cocoa butter, lanolin, coconut oil, sunflower oil, dimethicone, or any combination thereof. In some embodiments, the concentration of the moisturizing agent can be between about 0.01% to about 5%, about 0.01% to about 4%, about 0.01% to about 3%, about 0.01% to about 2%, about 0.01% and about 1.5%, about 0.5% to about 5%, about 1% to about 5%, about 2% to about 5%, about 3% to about 5%, about 4% to about 5%, about 0.05% to about 3%, about 0.05% to about 2%, and/or about 1% to about 2%.

In some embodiments, the antimicrobial composition or formulation can be in the form of a lotion or body lotion. In some lotion embodiments, the concentration of each of the sodium chlorite and sodium percarbonate can be between about 0.05% to about 0.75%, about 0.05% to about 0.7%, about 0.05% to about 0.6%, about 0.05% to about 0.5%, about 0.05% to about 0.4%, about 0.05% to about 0.2%, about 0.05% to about 0.15%, about 0.8% to about 0.2%, about 0.1% to about 0.15%, about 0.1% to about 0.75%, about 0.2% to about 0.75%, about 0.3% to about 0.75%, about 0.4% to about 0.75%, about 0.5% to about 0.75%, about 0.6% to about 0.75%, about 0.2% to about 0.7%, about 0.2% to about 0.6%, or about 0.3% to about 0.5% by weight. The shampoo, body wash, and face wash applications can also include one or more surfactants ranging in total concentration from about 2% to about 25%, about 2% to about 25% by weight of the solution. The lotion applications can also include an emollient, humectant, or other moisturizing agent including but limited to glycerin, shea butter, cocoa butter, lanolin, coconut oil, sunflower oil, dimethicone, or any combination thereof. In some embodiments, the concentration of the moisturizing agent can be between about 0.01% to about 10%, about 01% to about 9%, about 0.01% to about 8%, about 01% to about 7%, about 0.01% to about 6%, about 0.01% to about 5%, about 0.01% to about 4%, about 0.01% to about 3%, about 0.01% to about 2%, about 0.01% and about 1.5%, about 1% to about 10%, about 2% to about 10%, about 3% to about 10%, about 4% to about 10%, about 5% to about 10%, about 6% to about 10%, about 7% to about 10%, about 0.5% to about 5%, about 1% to about 5%, about 2% to about 5%, about 3% to about 5%, about 4% to about 5%, about 0.05% to about 3%, about 0.05% to about 2%, and/or about 1% to about 2% by weight of the solution. The lotion application can also include one or more emulsifier, including but limited to one or more of cetearyl alcohol, ceteareth-20, or glyceryl dilaurate, having a concentration of between about 1% to about 10%, about 2% to about 10%, about 3% to about 10%, about 4% to about 10%, about 5% to about 10%, about 6% to about 10%, about 7% to about 10%, about 0.5% to about 5%, about 1% to about 5%, about 2% to about 5%, about 3% to about 5%, about 4% to about 5%, about 2% to about 9%, about 3% to about 8%, about 4% to about 6% and/or about 1% to about 2% by weight of the solution.

### Methods of Use

The antimicrobial formulation can be used in many different types of applications or administration protocols. For instance, in some embodiments, the antimicrobial formulation is a hard surface disinfectant, and can come in a liquid form, which can be poured or sprayed from a conventional spray bottle onto a desired surface to be cleaned. In other embodiments, the antimicrobial formulation can come in aerosol form and be an air disinfectant. In some embodiments, the antimicrobial formulation can be an electrostatically sprayable air disinfectant. The anti-microbial formulation can be used to clean surfaces in a variety of environments, including but not limited to medical, industrial, and residential environments (kitchens, bathrooms, etc.), hospitals, medical facilities, medical clinics, schools or other public buildings, industrial packaging plants, factories, manufacturing facilities, food processing and packaging facilities, restaurants, bars, etc. The antimicrobial formulation can also be utilized as a wound cleaner or an antiseptic for cleaning out cuts, abrasions, or other wounds as well as to sterilize an injection or surgical site in a healthcare setting. The antimicrobial formulation can also be used for industrial cleaning services, such as for mold and mildew removal services. The antimicrobial formulation can also be used for cleaning HVAC applications for killing Legionella, biofilm and other organisms. The antimicrobial formulation can also be used for line or tubing cleaning such as in dialysis systems, and for beer, soda, and other fluid lines in a restaurant or commercial setting. The antimicrobial formulation can also be used as a n additive to a water supply for water purification purposes, such as in drinking water systems, medical water systems, etc. The antimicrobial formulation can also be used in air sanitation systems such as plug in diffusers to disperse the formulation into the air and clean and purify the air.

As described above, the formulations may be ready to use. In embodiments where the formulation is provided as a concentrate, it may be diluted by the user with additional water in an amount ranging from about 1:1 to about 1:40, about 1:1 to about 1:20, about 1:2 to about 1:20, about 1:2 to about 1:15, about 1:5 to about 1:20, about 1:5 to about 1:15, about 1:10, about 1:25, about 1:20, or about 1:40. In some embodiments, the concentrate is diluted with enough water to provide about 0.25% to about 1% of the chlorite by weight of the formulation, about 0.25% to about 1% by weight of the percarbonate by weight of the formulation, and about 0.1% to about 0.5% of the surfactant by weight of the formulation.

The formulations may be applied directly to the desired surface. In certain embodiments, a surface is saturated with the formulation. The formulation is allowed to contact the surface for a period of time sufficient to substantially kill pathogens on the surface, such as for about 10 minutes, about 5 minutes, about 2 minutes, about 1 minutes, or about 30 seconds. After this period of time, the surface may optionally be rinsed with water. Nevertheless, the formulations advantageously do not need to be rinsed for most applications.

For agricultural uses, the method comprises the steps of providing an antimicrobial formulation, including any of the above described formulations, and applying the anti-microbial formulation to the agricultural product. The antimicrobial formulation can effectively kill undesirable bacteria, viruses, and/or fungi from the agricultural product without damaging, discoloring, and/or altering the flavor of the agricultural product. In some embodiments, the agricultural product can be plants of various varieties, vegetables, fruits, legumes, grains, cannabis, hemp etc. Testing of one embodiment of the antimicrobial formulation on cannabis plants showed that the antimicrobial formulation provided the sporicidal efficacies discussed herein while no significant damage or negative effect was observed in the cannabis plants to which the compound was applied. In still other embodiments, the anti-microbial formulation can be applied to the meat and poultry industry to clean meat and poultry products prior to packaging. Animal living and production facilities also can be disinfected using the present formulations, including barns, pens, dairy production, egg production, and the like.

Another aspect of the present invention is a method of treating a food source, such as that to be fed to animals or livestock, comprising the steps of: providing any of the above-described antimicrobial formulations and applying the anti-microbial formulation on the food source. Applying the antimicrobial formulation to a food source such as animal feed products can help kill any unwanted bacteria, viruses and/or fungi prior to the food source being fed to the animal or livestock. Having the food source treated with the antimicrobial formulation of the present disclosure also may kill harmful bacteria inside the belly or digestive track of the target animal once the food source is ingested, including in chickens and pigs. Such a treatment protocol can help keep the animals or livestock healthy and help prevent unwanted bacteria to be passed on to humans who may consume any such animals or livestock.

Another aspect of the present disclosure is a method of treating a water supply including the steps of: providing an antimicrobial formulation as described above; and introducing the anti-microbial formulation into the water supply. In some embodiments, the antimicrobial formulation can meet the EPA standards for a Category IV product or be non-toxic and non-irritant from a regulatory standpoint. As such, the anti-microbial can be consumed safely by humans and animals such that the antimicrobial formulation can be used to treat drinking water supplies or other water supplies which can interact with humans and animals. In a medical setting, the antimicrobial formulation can be used to treat water supplies which can be provided to varying medical devices, e.g. a medical dialysis unit.

The antimicrobial formulation of the present disclosure can thus provide antimicrobial properties which can help kill unwanted bacteria from a surface or product. In some embodiments and application, the antimicrobial formulation can also help provide antibacterial, antifungal, sanitization, disinfectant, odor elimination, or other beneficial cleaning characteristics. The anti-microbial formulation can also generally be safe for contact with humans and animals, such that the product can be used to treat agricultural products and or water supplies which may be safely consumed or utilized by the public.

### EXAMPLES

### Example 1

The minimum inhibitory concentration of several compounds was tested and the results are summarized in Table 1. The MIC is the lowest concentration of an antimicrobial agent at which a test microorganism will not grow.

TABLE 1

| | * MIC against Pseudomonas |
|---|---|
| Antibacterial | aeruginosa ATCC 27853 |
| 0.5% Sodium Hypochlorite | 1:4 |
| 0.5% Chlorine Dioxide | 1:32 |
| 0.5% Sodium Chlorite | 1:8 |
| 0.5% Stepan BTC 2125M | 1:32 |
| 0.5% Sodium Percarbonate | 1:8 |
| 0.5% Sodium Chlorite and | 1:64 |
| 0.5% Sodium Percarbonate | |
| 0.5% Sodium Chlorite and | 1:32 |
| 0.5% Sodium Percarbonate | |
| with buffer | |
| 0.5% Peracetic Acid | 1:64 |

The lower the MIC dilution, the more effective a antimicrobial agent is at killing a microbe. As seen in Table 1, sodium chlorite alone has a similar MIC to sodium percarbonate. When sodium chlorite and sodium percarbonate are combined, they appear to have more than an additive effect, and may act synergistically in combination. While not being bound by theory, Pseudomonas sp. are much more resistant to disinfectants than other bacteria (Rutala W A, Cole E C (1987) Ineffectiveness of hospital disinfectants against bacteria: a collaborative study. Infect. Control. 8:501-506; Favero M S (2004) Naturally occurring microrganisms and their resistance to physical and chemical agents. In: Rutala W A, ed. Disinfection, sterilization and antisepsis: Principles, practices, challenges, and new research. Washington DC: Association for Professionals in Infection Control and Epidemiology. 1-14; Rutala W A, Weber D J (2008) Guideline for Disinfection and Sterilization in Healthcare Facilities. Centers for Disease Control and Prevention.).

The protocol for the above noted determination of the minimal inhibitory concentration (MIC) of antimicrobials is provided in more detail herein. This protocol has been designed to accurately determine the killing of microorganisms by antimicrobial agents, since this is the guarantee most often used in preventative products, such as disinfectants and sanitizers, for legal purposes. In a standard MIC assay a growing culture of 1,000,000 or 10⁶ microorganisms is subjected to serial 1:2 (½) dilutions of the preventative agent and then their growth is observed 16 to 24 hours later. A 99.9% killing is observed if only 1000 or 103 microorganisms remain. A 99.99% killing is observed if only 100 or 10² microorganisms remain. The protocol, is as follows:
Day one: Prepare 2 mL overnights in 1× media, for example, 1× tryptic soy broth (TSB), of the microorganisms to be tested.
Day two: The ½ dilution of the antimicrobial to be tested is prepared by diluting 5 mL of the original antimicrobial solution to be diluted with 5 mL of dH2O and thoroughly mixing the resulting solution. The ¼ dilution is prepared by diluting 5 mL of the ½ diluted solution with 5 mL of dH2O and thoroughly mixing the resulting solution. ⅛, 1/16, 1/32, 1/64, 1/128, 1/256 and 1/512 dilutions are also prepared. A saturated 1× TSB overnight of the microorganism to be tested is diluted to 1,000,000 or 10⁶ colony forming units in 2× TSB. Pseudomonas aeruginosa strains typically yield saturated overnights that contain about 1×10¹⁰ CFU per mL. 1 mL of the diluted overnight is added to 1 mL of each of the dilutions of the antimicrobial agent to be tested, ranging from ½ to 1/1024, in culture tubes. Note that the ½ dilution of the antimicrobial agent yields a final ¼ dilution when added to the 1 mL of diluted overnight, etc. . . . . The final concentration of bacteria that is tested is 1,000,0000 or 1×10⁶ CFU. The cultures are then aerated at the appropriate temperature for 16 to 24 hours.
Day three: The tubes are checked for growth to determine what are the highest dilutions of antimicrobial agent that prevents the growth of the microorganisms. These are noted and recorded. The dilution that does not inhibit growth and the next two lower dilutions that inhibit growth are also noted and recorded. The highest dilution that yields complete killing of the microorganism and the next two lower dilutions are further evaluated by plating 100 ul of the overnight solution onto the appropriate plate, for example 1× TSB plates. Once the spread media has dried, the plates are incubated at the proper temperature overnight.
Day four: Whether dilutions that appeared not to have any growth in broth actually prevented growth are noted. Only a dilution that showed no growth in broth and yielded no colonies when plated is a dilution that truly prevented microbial growth.

1. An aqueous antimicrobial formulation comprising an aqueous solution comprising a
chlorite salt in a concentration ranging from about 0.05% to about 25% by weight of the aqueous solution, a percarbonate salt in a concentration ranging from about 0.05% to about 25% by weight of the aqueous solution, and a surfactant in a concentration ranging from about 0.5% to about 50% by weight of the aqueous solution.

2. The antimicrobial formulation of claim 1, wherein the chlorite salt is chosen from ammonium chlorite, calcium chlorite, magnesium chlorite, potassium chlorite, sodium chlorite, and any combination thereof.

3. The antimicrobial formulation of claim 2, wherein the chlorite salt comprises the sodium chlorite.

4. The antimicrobial formulation of claim 1, wherein the concentration of the chlorite salt ranges from about 0.25% to about 1%, 0.25% to about 0.9%, 0.25% to about 0.8%, 0.25% to about 0.7%, 0.25% to about 0.6%, 0.25% to about 0.5%, about 0.5% to about 0.9%, about 0.5% to about 0.8%, about 0.5% to about 0.7%, about 0.5% to about 0.6%, about 0.05% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20% about 10% to about 15%, or about 15% to about 20% by weight of the aqueous solution.

5. The antimicrobial formulation of claim 1, wherein the percarbonate salt is chosen from ammonium percarbonate, calcium percarbonate, magnesium percarbonate, potassium percarbonate, sodium percarbonate, and any combination thereof.

6. The antimicrobial formulation of claim 5, wherein the percarbonate salt comprises sodium percarbonate.

7. The antimicrobial formulation of claim 1, wherein the concentration of the percarbonate salt ranges from about 0.25% to about 25% about 0.25% to about 1%, 0.25% to about 0.9%, 0.25% to about 0.8%, 0.25% to about 0.7%, 0.25% to about 0.6%, 0.25% to about 0.5%, about 0.5% to about 0.9%, about 0.5% to about 0.8%, about 0.5% to about 0.7%, about 0.5% to about 0.6%, about 0.05% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20% about 10% to about 15%, or about 15% to about 20% by weight of the aqueous solution.

8. The antimicrobial formulation of claim 1, wherein the surfactant comprises lauramine oxide, sodium dodecylbenzene sulfoate, sodium decyl sulfate, sodium docecyl sulfate, sodium octyl sulfonate, sodium xylene sulfonate, magnesium lauryl sulfate, sodium lauryl sulfate, C₉-C₁₁ ethoxylated alcohols, or any combination thereof.

9. The antimicrobial formulation of claim 8, wherein the surfactant comprises sodium docecyl sulfate, sodium dodecylbenzene sulfonate, C₉-C₁₁ ethoxylated alcohols, or a combination thereof.

10. (canceled)

11. (canceled)

12. (canceled)

13. The antimicrobial formulation of claim 1, wherein the concentration of the surfactant ranges from about 0.05% to about 50%, about 0.05% to about 1%, 0.05% to about 0.9%, 0.05% to about 0.8%, 0.05% to about 0.7%, 0.05% to about 0.6%, 0.05% to about 0.5%, about 0.05% to about 0.4%, about 0.05% to about 0.3%, about 0.05% to about 0.2%, about 0.05% to about 0.1%, about 0.1% to about 1%, 0.1% to about 0.9%, 0.1% to about 0.8%, 0.1% to about 0.7%, 0.1% to about 0.6%, 0.1% to about 0.5%, about 0.1% to about 0.4%, about 0.1% to about 0.3%, about 0.1% to about 0.2%, about 1% to about 25%, about 5% to about 25%, about 10% to about 25%, about 15% to about 25%, about 20% to about 25%, about 1% to about 20%, about 1% to about 15%, about 1% to about 10%, about 1% to about 5%, about 5% to about 20%, about 5% to about 15%, about 5% to about 10%, about 10% to about 20%, about 10% to about 15%, about 15% to about 20%, about 1% to about 50%, about 5% to about 50%, about 10% to about 50%, about 15% to about 50%, about 20% to about 50%, or about 25% to about 50% by weight of the aqueous solution.

14. The antimicrobial formulation of claim 1, further comprising a stabilizer in an amount ranging from about 10 mM to about 2500 mM, wherein the stabilizer comprises an acetate salt, a bicarbonate salt, a carbonate salt, a metasilicate salt, a citrate salt, or any combination thereof.

15. (canceled)

16. (canceled)

17. The antimicrobial formulation of claim 14, wherein the stabilizer comprises potassium acetate, sodium acetate, potassium bicarbonate, sodium bicarbonate, potassium carbonate, sodium carbonate, potassium metasilicate, sodium metasilicate, or any combination thereof.

18. The antimicrobial formulation of claim 17, wherein the stabilizer comprises sodium bicarbonate, sodium carbonate, or a combination thereof.

19. The antimicrobial formulation of claim 14, wherein the concentration of the stabilizer ranges from about 5 mM to about 90 mM, about 5 mM to about 80 mM, about 5 mM to about 70 mM, about 5 mM to about 60 mM, about 5 mM to about 50 mM, about 5 mM to about 40 mM, about 5 mM to about 30 mM, about 5 mM to about 20 mM, about 10 mM to about 90 mM, about 10 mM to about 80 mM, about 10 mM to about 70 mM, about 10 mM to about 60 mM, about 10 mM to about 50 mM, about 10 mM to about 40 mM, about 10 mM to about 30 mM, about 10 mM to about 20 mM, about 20 mM to about 100 mM, about 20 mM to about 90 mM, about 20 mM to about 80 mM, about 20 mM to about 70 mM, about 20 mM to about 60 mM, about 20 mM to about 50 mM, about 20 mM to about 40 mM, about 20 mM to about 30 mM, about 30 mM to about 100 mM, about 30 mM to about 90 mM, about 30 mM to about 80 mM, about 30 mM to about 70 mM, about 30 mM to about 60 mM, about 30 mM to about 50 mM, about 30 mM to about 40 mM, about 40 mM to about 100 mM, about 40 mM to about 90 mM, about 40 mM to about 80 mM, about 40 mM to about 70 mM, about 40 mM to about 60, about 40 mM to about 50 mM, about 50 mM to about 100 mM, about 50 mM to about 90 mM, 50 mM to about 80 mM, 50 mM to about 70 mM, 50 mM to about 60 mM, 60 mM to about 100 mM, 60 mM to about 90 mM, 60 mM to about 80 mM, 60 mM to about 70 mM, 70 mM to about 100 mM, 70 mM to about 90 mM, 70 mM to about 80 mM, 80 mM to about 100 mM, 80 mM to about 90 mM, or 90 mM to about 100 mM, about 5 mM to about 2500 mM, about 10 mM to about 2500 mM, about 100 mM to about 2500 mM, about 500 mM to about 2500 mM, about 1000 mM to about 2500 mM, or about 2000 mM to about 2500 mM.

20. (canceled)

21. The antimicrobial formulation of claim 1, further comprising a buffer comprising acetic acid, citric acid, sodium acetate, potassium acetate, sodium carbonate, potassium carbonate, sodium bicarbonate, potassium bicarbonate, sodium citrate, potassium citrate, or any combination thereof.

22. The antimicrobial formulation of claim 1, wherein the formulation has a pH ranging from, about 8.5 to about 9.5.

23. (canceled)

24. The antimicrobial formulation of claim 1, further comprising at least one of a fragrance, a colorant, a sudsing agent, an aerosolizing agent, or a thickener.

25. The antimicrobial formulation of claim 1, wherein the formulation comprises about 0.25% to about 1% of the chlorite by weight of the formulation, about 0.25% to about 1% by weight of the percarbonate by weight of the formulation, and about 0.1% to about 0.5% of the surfactant by weight of the formulation.

26. The antimicrobial formulation of claim 1, wherein the formulation comprises about 10 to about 25% of the chlorite by weight of the formulation, about 10% to about 25% by weight of the percarbonate by weight of the formulation, and about 5% to about 25% of the surfactant by weight of the formulation.

27.-32. (canceled)

33. The antimicrobial formulation of claim 1, wherein the concentration of produced chlorine dioxide in solution is less than 0.01% by weight of the aqueous solution.

34.-41. (canceled)
